(19) United States
(12) Patent Application Publication
(10) Pub. No.: US 2022/0039570 A1
(43) Pub. Date: Feb. 10, 2022

(54) AWAKENING APPARATUS

(71) Applicant: JayShaud Potter, Battle Creek, MI (US)

(72) Inventor: JayShaud Potter, Battle Creek, MI (US)

(21) Appl. No.: 17/393,612

(22) Filed: Aug. 4, 2021

Related U.S. Application Data
(60) Provisional application No. 63/060,737, filed on Aug. 4, 2020.

(51) Int. Cl. A47G 9/10; A47G 9/00; A61B 5/00; H04W 4/80

(52) U.S. Cl. CPC A47G 9/1045; A47G 9/007; A61B 5/4809; A47G 2200/20; H04W 4/80; A47G 2009/005; A61B 5/4812

(57) ABSTRACT

An apparatus for alerting a sleeping subject, the apparatus has a wireless device that stores an application program configurable for control of the apparatus; and a pillow that is configured to be in signal communication with at least the wireless device. The pillow has a charge storage source for storing electrical energy; a control processor that is configured with a set of encoded instructions for maintaining communication between the wireless device and the pillow; and one or more actuators configured to receive a signal from the control processor and respond to provide an awakening stimulus to the sleeping subject.

### CROSS REFERENCE TO RELATED APPLICATIONS

This application claims the benefit of U.S. Provisional Application Ser. 63/060,737, entitled “AWAKENING APPARATUS”, in the name of JayShaud Potter, filed Aug. 4, 2020 and incorporated herein in its entirety.

### TECHNICAL FIELD

The present disclosure relates generally to alarm devices and more particularly to a system using a pillow that is configured to stimulate a reclining person to an awakened state, such as under emergency conditions.

### BACKGROUND

A sleeping person may need to be alerted to any of a number of different urgent situations that require awakening, attention, and action. These can include national, regional, or local area alerts or emergency phone calls that are provided over a telephone or other wireless connection. In addition, alert situations can originate from various sensors within a house or building, or from a motor vehicle, boat, or other device, including smoke detectors, carbon monoxide detectors, break-in alarms, security sensors, open door sensors, door bells, child or infant monitors, moisture detectors, monitoring devices for the elderly or those with chronic or other life-threatening conditions, and like devices.

Conventional devices that serve to alarm and awaken the sleeper often rely on audible alarm devices, such as devices that ring a bell, beep, or otherwise emit sound that is intended to arouse the sleeper quickly and stimulate quick response. However, there is an appreciable percentage of the population that sleep so soundly or have at least some degree of hearing impairment, so that audible alarms and alert devices are wholly or partially ineffective for awakening a sleeper. The inability to perceive or to be stimulated by audible warning signals and alerts can put those with hearing impairment at particularly high risk, during sleep, in the event of national or local emergency, where dangerous weather conditions are imminent, in danger from fire or flooding, or where safety and security of the home or property may otherwise be compromised.

For the subject who is suddenly awakened by an alarm device, it can be difficult to determine the cause and source of the alarm situation. With conventional alarm systems and devices, the subject must recognize the source of a problem by foreknowledge of audible aspects of the alarm, such as alarm pitch, location, undulation, etc. It can take a subject some amount of time for orientation to surroundings and for suitable awareness and identification of a particular problem and of what action may be required. This situation can be more troubling for some subjects, who may suffer confusion or panic when jarred awake to an emergency situation.

There is, then, a need for an alerting device that can provide some amount of information useful to the person awakened, that allows some flexibility and adaptability to user preferences, and that meets particular needs of those with hearing or other sensory impairment.

### SUMMARY

It is an object of the present disclosure to address the need for an alerting device that addresses at least some of the problems noted previously in the background section. Embodiments of the present disclosure provide an alerting device that can have multiple sensory outputs, that allows some degree of programmable behavior, that communicates wirelessly with smartphones, tablets, and other personal communications devices, and that has a configuration that is likely to be used by a subject who intends to sleep.

According to an aspect of the present disclosure, there is provided an apparatus for alerting a sleeping subject, the apparatus comprising:
a wireless device that stores an application program configurable for control of the apparatus; and
a pillow that is configured to be in signal communication with at least the wireless device, the pillow comprising;
(a) a charge storage source for storing electrical energy;
(b) a control processor that is configured with a set of encoded instructions for maintaining communication between the wireless device and the pillow;
(c) one or more actuators configured to receive a signal from the control processor and respond to provide an awakening stimulus to the sleeping subject.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic diagram that shows some uses and types of non-auditory alarms that can be provided by a pillow.

FIG. 2 is a diagram that shows a number of example displays that can appear to the awakened subject.

FIG. 3 is a schematic diagram showing components within a pillow configured for use with the system shown in FIG. 1.

FIGS. 4A and 4B show pillow components from side and top views, with various arrangements of vibration and lighting components.

FIGS. 5A-5D portray various screens that show various parts and functions of an app for system configuration and control.

### DETAILED DESCRIPTION OF EXEMPLARY EMBODIMENTS

An embodiment of the present disclosure addresses the need for an alerting device for awakening a sleeper by energizing one or more non-auditory output devices that are housed within a pillow used by the sleeper.

Where they are used, the terms “first”, “second”, and so on, do not necessarily denote any ordinal, sequential, or priority relation, but are simply used to more clearly distinguish one element or set of elements from another, unless specified otherwise.

As used herein, the term “energizable” relates to a device or set of components that perform an indicated function upon receiving power and, optionally, upon receiving an enabling signal.

In the context of the present disclosure, the term “coupled” is intended to indicate a mechanical association, connection, relation, or linking, between two or more components, such that the disposition of one component affects the spatial disposition of a component to which it is coupled. For mechanical coupling, two components need not be in direct contact, but can be linked through one or more intermediary components.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

In the context of the present disclosure, the terms “subject” and “user” are considered to be equivalent and refer to the person sleeping and using or configuring the apparatus of the present invention.

In the context of the present disclosure, the term “actuator” applies to a type of transducer that, in response to a received signal, generates light, motion, vibration, sound, air flow, scent emission, or other form of energy or stimulus that can be perceived by the senses.

The phrase “in signal communication” as used in the application means that two or more devices and/or components are capable of communicating with each other via signals that travel over some type of signal path. Signal communication may be wired or wireless. The signals may be communication, power, data, or energy signals which may communicate information, power, and/or energy from a first device and/or component to a second device and/or component along a signal path between the first device and/or component and second device and/or component. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

As noted in the background section given previously, for people who are unresponsive to audible alarm devices, whether through hearing impairment, infirmity, exhaustion, medication, or for particularly deep sleepers, there can be increased risk in various situations where conventional audible alarms would otherwise be adequate. Embodiments of the present disclosure are directed to the hearing-impaired, as well as to others who, for any reason, may not be suitably stimulated by audible alarms and prefer other non-auditory methods of stirring them from sleep when there is a perceived need.

In order to provide an alarm device that is likely to be able to stimulate and awaken the sleeper, embodiments of the present disclosure use a pillow as a housing for internal components, with apparatus behavior configured and adapted for preferences of the individual subject and in signal communication with external devices for receiving relevant information. Although the embodiment described in detail relates to a device that is primarily intended for the hearing-impaired, it should be noted that the disclosed device can be designed and configured for use by other segments of the population. The programmability of device response allows ready adaptation for anyone who would benefit from enhanced stimulation for awakening, including parents, those caring for the elderly or others who may be on various types of monitoring or support systems, emergency personnel, patients recovering from trauma or surgery, etc.

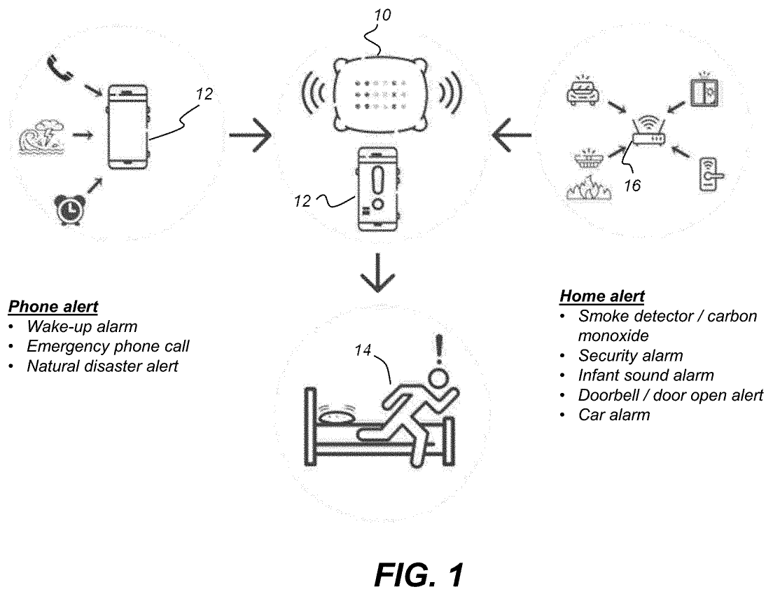

FIG. 1 is a schematic diagram that shows some uses and types of non-auditory alarms that can be provided from within a pillow 10. Phone alerts can be received in response to a personal wake-up alarm, such as an alarm programmed at the user interface to a smart phone 12, a tablet or other personal communications device. Other types of alarms can include weather or other natural disaster alerts received at a smart phone or tablet, such as for high winds, ice storm, hurricane or tornado conditions, volcanic activity, earthquake, or other natural disaster alerts. The subject 14 may also receive emergency phone calls or notifications, such as for police, fire, medical assistance, or volunteer personnel, for example.

FIG. 1 also shows various home alerts that can be received, including alert signals from smoke or carbon monoxide detectors, security sensors, infant or child monitors, doorbell or entryway alerts, car alarms, etc. As shown in FIG. 1, devices in signal communication with pillow 10 circuitry can include a wired or wireless device such as a smart phone 12, a tablet, a wireless transceiver 16, or any suitably equipped personal communications, alarm, or sensing device. Any type of “smart home” technology sensor or reporting device can provide a signal that prompts an alert response from pillow 10. Bluetooth, WiFi, or other wireless communication mechanism can be used for signal communication with smart phone 12, tablet, smart watch, personal communications device, or other system that acts as a “wireless device” in the context of the present disclosure.

Smart phone 12 can be used to provide signals to pillow 10, as indicated in FIG. 1, such as signals related to emergency alarms or urgent phone calls from selected sources that are in signal communication with smart phone 12. In some embodiments, pillow 10 can act as a type of output peripheral device to smart phone 12. In addition, an application that executes on phone 12 can also be used to program pillow 10 behavior and to configure wireless connections with other devices, such as with automobile or home security systems, for example. Transmission between pillow 10 and phone 12 or between pillow 10 and other systems or devices can use a standard radio frequency (RF) wireless protocol, such as Bluetooth protocol, accessible to a wide range of handheld communications devices as well as to a range of desktop and instrumentation systems. Alternately, other wireless mechanisms can be used, including ultrasound and optical signals, for example.

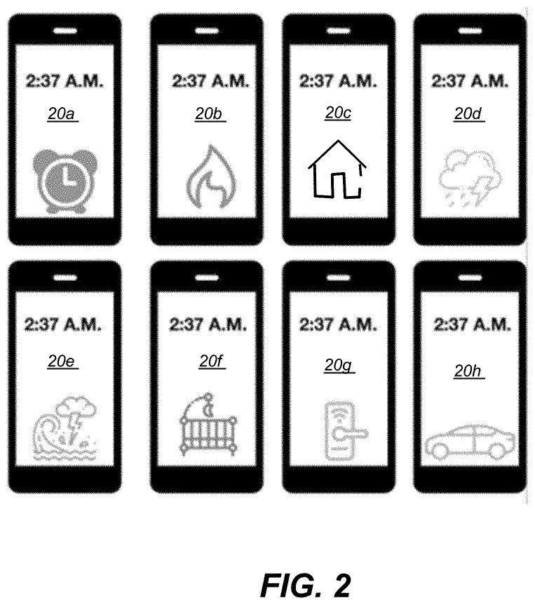

Smart phone 12 can also serve to display information to the awakened subject, such as indicating the source of an alarm or other condition that causes activation of the pillow. FIG. 2 is a diagram that shows a number of example displays that can appear to the awakened subject, each indicating a possible source of an alert or warning condition. A display 20a can indicate awakening due to an alarm clock setting. A display 20b can indicate fire or smoke detection. A display 20c can indicate a security system alert, such as a potential break-in or unlocked door or window. A display 20d can indicate threatening or severe weather. A display 20e can indicate possible flooding. A display 20f can indicate signals from an infant or child monitor. A display 20g can indicate an unlocked door or an attempt to open an outside door. A display 20h can indicate a car alarm signal. Other sources can be provided for communication with smart phone 12 and actuation of pillow 10 response.

### Pillow 10 Components

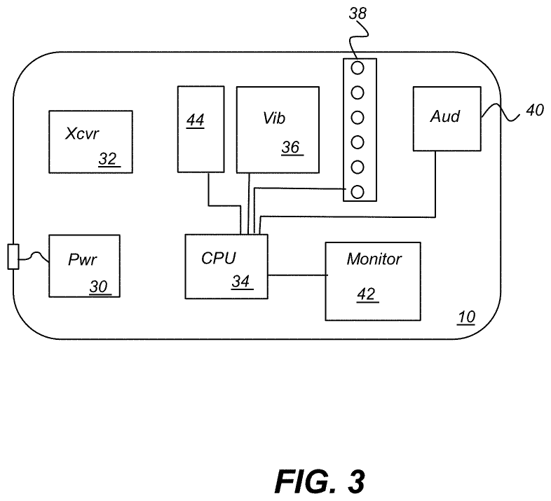

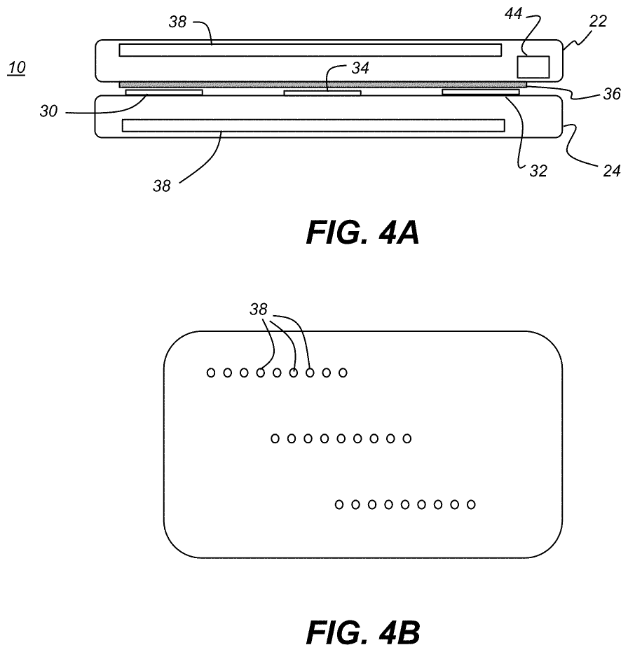

FIG. 3 is a schematic diagram showing components within a pillow 10 configured for use with the system shown in FIG. 1. FIGS. 4A and 4B show different views for exemplary component arrangements. The pillow itself can be a foam pillow that utilizes a memory foam substrate, for example. An internal battery 30 or other charge storage source can provide a rechargeable power source, allowing pillow 10 to be moved to a suitable position for the sleeper. Alternately, a separate power cord can provide external power, such as from a power or directly from an electrical outlet. A wireless transceiver 32 can provide signal communications with a personal communications device such as a smart phone or with other wireless components, such as might be used with a home security or alarm system. A processor 34 can be programmed with sufficient logic and computer instructions to communicate with external devices and to operate one or more actuators that convert stored power to sensible (sensory) output for stimulating and awakening the sleeping subject.

For the purpose of description, three specific types of actuators are shown for providing the sensory output: a vibrator 36 that generates a low-frequency mechanical vibration; a light source 38 that can illuminate one or more light emitters in an array as a warning indicator; and an optional speaker 40 that be used, such as to supplement the alarm for the subject or for others who may be nearby, for example. Other actuators 44 can include devices that blow a current or jet of air, generate a smell, cause a rocking motion, modify pillow softness or other characteristic, or generate other stimuli to awaken or alert the subject.

FIG. 4A shows a transverse or cross-sectional view of pillow 10 according to an embodiment. Foam layers 22 and 24 encase various internal components of pillow 10, making the device more user-friendly and providing some protection of components. Vibrator 36 can extend as a single strip between two layers of foam 22 and 24. Alternately, multiple strips or vibrators 36 can be provided, positioned at suitable locations within pillow 10. Light sources 38 can be positioned nearer the surface of pillow 10, such as along or just within the top and bottom foam layers 22, 24.

FIG. 4B shows one of a number of possible arrangements for light sources 38 in pillow 10. LED light sources can be used, with various dot and strip combinations, to provide light from broad areas of pillow 10 surface. Lights can be energized simultaneously or in sequence, continuously on or pulsing, for example. Light color can change during display. Light color and behavior (such as intensity, flashing speed or color sequencing, for example) can also be programmed to indicate particular urgent conditions. Light can be programmed or otherwise configured to display as discrete dots or continuous strips.

It can be appreciated that the configurations shown in FIGS. 3, 4A, and 4B are exemplary only, and admits any of a number of improvements, enhancements, and variants, within the scope of the present disclosure. Thus, for example, pillow 10 can have only a partial subset of the actuators shown, such as only having vibrator 36 or equivalent sensory output or having only light sources 38 and vibrator 36. Similarly, two or more vibrators 36 can be provided, at different locations within pillow 10, for example. Or, multiple light arrays can be provided. Setup of pillow 10 response can be hard-wired or programmed by the user, as described subsequently, to allow selection of output behavior.

An optional sleep monitor 42 can be provided, with appropriate sensors and logic for tracking and reporting subject sleep states. The sleep state detection can be used, for example, to adjust intensity or energy level of actuation for one or more actuators within pillow 10.

### Application

The awakening apparatus can be manufactured or pre-programmed to have fixed behavior in response to various conditions. However, an advantageous embodiment of the present disclosure has an associated application or downloadable application or “app” that allows the behavior of pillow 10 to be adjusted and configured in a number of ways in order to suit the particular requirements of a subject.

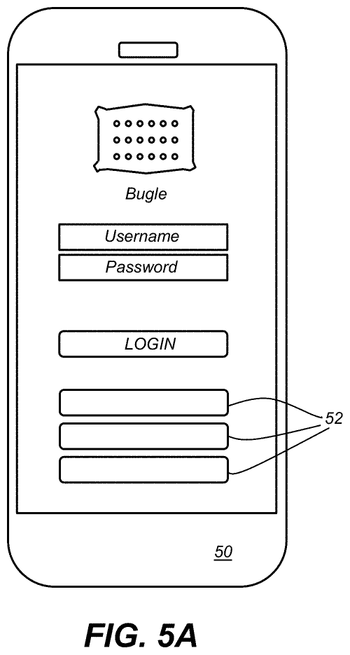

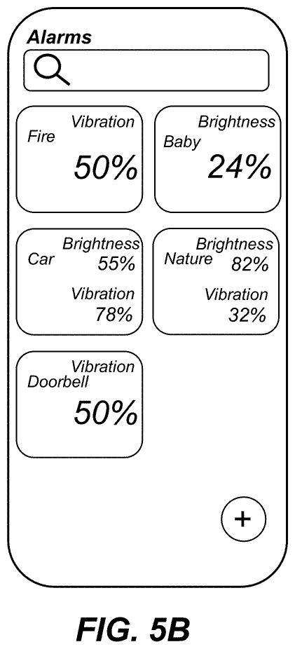

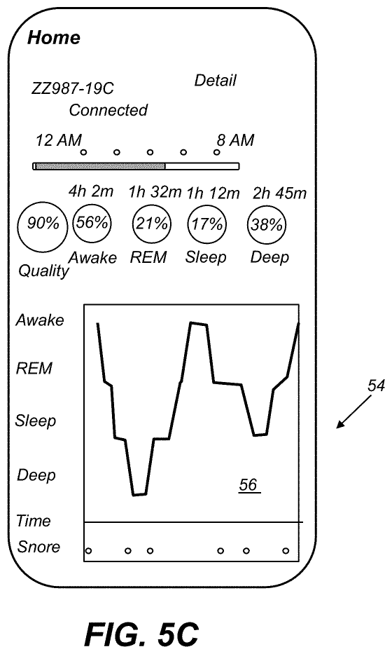

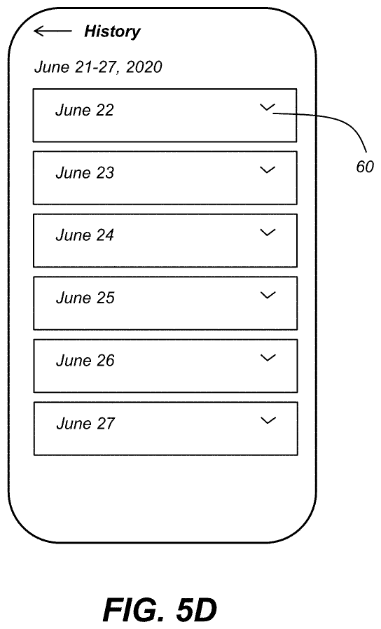

By way of example, FIGS. 5A-5D portray various screens that show various parts and functions of an app for pillow 10 configuration and control. FIG. 5A shows a login screen 50 for an application, appropriately named Bugle, for pillow 10 configuration and control. Login and account security allows a measure of privacy and individual preference for customization parameters. Other buttons 52 enable transition to other functions of the mobile phone.

FIG. 5B shows an exemplary control screen that allows system response to be customized for various types of input signals and sensed conditions. Different actuators can be configured for different emergency/urgency conditions. As shown in this example, vibration settings can be individually adjusted, based on category of urgency or severity, likelihood, or other user-selected criteria. Examples for adjustment related to alarm or alert conditions include:
(i) vibration adjustments, such as intensity or amplitude, frequency, pulsing, location (where more than one vibrator 36 is deployed);
(ii) light adjustments, including brightness, duration, flashing, color, color shifting;
(iii) audible adjustment, including volume, content.
   Other types of adjustment can be applied. For example, adjustment can accentuate changes in air current or pillow softness characteristics.

By way of example, some users may find that light is sufficient for an alert, with vibration disabled; others may determine that vibration is most appropriate, and light not useful or annoying; thus, light may be independently disabled. Still other users may want both light and vibration, with various patterns or pulsing, for example, and may program pillow 10 accordingly.

### Sleep Statistics Reporting

As noted previously, sleep quality data can be provided. Sleep monitoring provided by pillow 10 sensor data, can use known methods for identifying sleep states and duration.

FIG. 5C shows an exemplary display screen for reporting results from sleep monitoring. Sleep data 54 can be obtained using monitor 42, as described with reference to FIG. 3. Various sleep states and duration within each state can be displayed, along with percentage time and other sleep quality indications. In the example shown, a graph 56 displays to show the sequence and duration of various sleep states. According to an embodiment, the level of intensity for an actuator can be responsive to the sleep state of the subject. Thus, for example, if pillow 10 sensors detect that the subject is in a deep sleep state, vibration levels can be accentuated, or light behavior can be more rapid.

FIG. 5D shows an exemplary display screen for displaying stored data, such as history of sleep data for each day, as well as alert data for recording when pillow 10 features for awakening may have been actuated. A pull-down menu 60 can be provided for detailed information on each stored date.

The battery or other charge storage source can be plugged into a standard charger, such as a USB charging port, in order to store sufficient charge for operation during the subject's sleep.

An apparatus for alerting a sleeping subject, has a wireless device that stores an application program configurable for the apparatus; and a pillow that is configured to be in signal communication with at least the wireless device. The pillow has (a) a charge storage source for storing electrical energy; (b) a control processor that is configured with a set of encoded instructions for maintaining communication between the wireless device and the pillow; and (c) one or more actuators configured to receive a signal from the control processor and respond to provide an awakening stimulus to the sleeping subject.

1. An apparatus for alerting a sleeping subject, the apparatus comprising:
a wireless device that stores an application program configurable for control of the apparatus; and
a pillow that is configured to be in signal communication with at least the wireless device, the pillow comprising;
(a) a charge storage source for storing electrical energy;
(b) one or more actuators configured to provide an awakening stimulus to the sleeping subject in response to an enabling signal;
(c) a control processor that is configured with a set of encoded instructions for generating one or more enabling signals to the one or more actuators within the pillow.

2. The apparatus of claim 1 wherein the one or more actuators emits light in response to the enabling signal.

3. The apparatus of claim 1 wherein the one or more actuators vibrates in response to the enabling signal.

4. The apparatus of claim 1 wherein the one or more actuators emits a jet of air in response to the enabling signal.

5. The apparatus of claim 1 wherein the one or more actuators emits a smell in response to the enabling signal.

6. The apparatus of claim 1 wherein the wireless device is a mobile phone.

7. The apparatus of claim 1 wherein the apparatus detects and stores sleep data for the subject

8. The apparatus of claim 7 wherein one or more actuators have an adjusted intensity according to a detected sleep state of the subject.

9. An apparatus for alerting a sleeping subject, the apparatus comprising:
a wireless device that stores an application program configurable for control of the apparatus; and
a pillow that is configured to be in signal communication with at least the wireless device, the pillow comprising;
(a) a charge storage source for storing electrical energy;
(b) one or more actuators configured to provide an awakening stimulus to the sleeping subject in response to an enabling signal;
(c) a control processor that is configured with a set of encoded instructions for generating one or more enabling signals to the one or more actuators within the pillow;
(d) at least one sensor that is configured to detect a sleep state of the subject;
wherein the sleep state of the subject conditions an intensity of the awakening stimulus from the one or more actuators.

10. A method for alerting a sleeping subject, the method comprising:
configuring a wireless device for signal communication with components housed within a pillow, wherein the pillow has at least one actuator configured to provide an awakening stimulus to the sleeping subject in response to an enabling signal from the wireless device;
sensing a subject sleep state and reporting the sensed sleep state to the wireless device;
and
energizing the at least one actuator to provide the stimulus at a variable energy level, according to the sensed sleep state.
